(19) United States
(12) Patent Application Publication
(10) Pub. No.: US 2023/0149495 A1
(43) Pub. Date: May 18, 2023

(54) MODULATION OF NCRNAS, LYMPHOCYTES, NEUTROPHILS, INFLAMMASOMES AND P53

(71) Applicant: MUNISEKHAR MEDASANI, Hyderabad (IN)

(72) Inventors: Munisekhar MEDASANI, Hyderabad (IN); Renu MEDASANI, Madison, MS (US); Roshny MEDASANI, Hyderabad (IN)

(73) Assignee: Munisekhar MEDASANI, Hyderabad, TS (IN)

(21) Appl. No.: 18/099,118

(22) Filed: Jan. 19, 2023

Related U.S. Application Data
(63) Continuation-in-part of application No. PCT/IB2021/056492, filed on Jul. 19, 2021.
Application No. PCT/IB2021/056492 is a continuation of application No. 17/378,741, filed on Jul. 18, 2021, now abandoned.
(60) Provisional application No. 63/054,205, filed on Jul. 20, 2020.

(51) Int. Cl. A61K 36/68; A61K 45/06

(52) U.S. Cl. CPC A61K 36/68; A61K 45/06; A61K 2236/13; A61K 2236/15; A61K 2236/17; A61K 2236/35; A61K 2236/37

(57) ABSTRACT

Composition for modulation/regulation ncRNAs, Lymphocytes, Neutrophils, Inflammasomes and p53 for prevention, treatment and management of immune dysregulation, inflammation dysregulation and energy dysregulation, caused by respiratory viruses (COVID19, Influenza etc), or leading to Acute Respiratory Distress Syndrome (ARDS), Sepsis and Long Covid, immune senescence.

### BACKGROUND OF INVENTION

Present understanding is that Human body contains about 30-40 trillion cells and replaces about 15 billion cells every day, which is huge task and processes has to work very efficiently and well balanced to create new healthy cells and maintain old all the time. Every day human produces about 100 billion neutrophils, that tell how perfect that system has to run.

Though the nexus of Immunity-Inflammation-Energy dysregulation is being noticed in many diseases like Cardio Vascular Diseases (CVD), Alzheimer's(ALZ), Cancers (CAN), it is becoming more evident in SARS-CoV-2(COVID19). Difference between mild disease with faster recovery and hospitalisation/organ damage/death is dysregualation of immunity-inflammation-energy levels. Reduction of CD4, CD8 and increase of Neutrophils indicates immune dysregulation. Increase of IL-6, CRP, N-L-Ratio indicates inflammation dysregulation. Difficulty in breathing (Hypoxia) causes energy dysregulation and organ dysfunction leads to Cytokine storm, ARDS, SEPSIS and hospitalisastion.

Metabolic Dysregulation causes liver diseases and diabetes etc. Sometimes those progresses into next level with dysregulation of immunity and inflammation. For example NAFL to NASH to Cirrhosis to HCC, in this process insulin resistance, IL6 upregulation and immune dysregulation noted. Liver Diseases like FLD, NAFLD/MAFLD, AFLD, NASH, Cirrohosis becoming major burden in the western world with ˜25% of the population affecting with those. Metabolic Disorders like Diabetes, obesity and related diseases affecting about ⅓^{rd} of the population. Which becoming major burden.

These causes insulin sensitivity which further causes dysregulation in immunity and inflammation.

One of the main causes for metabolic disorders is excess eating, sedentary life style and increased levels of stress. These are causing inflammation which triggers immune dysregulation and causing many other diseases like Arthritis, Atherosclerosis, dyslipidemia, CVD, Vascular/neurovascular diseases.

Immune Dysregulation can happen due to external causes or internal causes like aging. Once immune dysregulation happens human body loses its ability to fight back on external pathogens like novel-viruses, sepsis causing bacteria, funguses and other diseases like like Cancers (eg. HCC etc).

Regulating non-coding RNAs (like miR155 & miR223 etc), interferon-a & -b (IFN-a/b),TNF-alpha, IL-6 and genes like p53 can regulate immunity, reduce inflammation, prevent/treat many types of cancers and delay aging. This also expected to revive functionality of organs (like liver etc) and glands (like thymus etc).

Due to aging there is an increased burden of inflammation and Immunosenescence. With increased health care and life expectancy we are able to extend the longevity of the humans, but unable to improve their health due to burden of inflammation and Immunosenescence. Managing Neuroinflammation becoming biggest hurdle for treatment of neurodegenerative diseases like ALZ, MS, PD, ALS etc.

In recent times COVID19 which already killed about 4 mill in 18 months is the known example to understand how delicate is the balance between immunity-inflammation-energy.

Previously existed low immunity (like aging) or low immunity caused by COVID19 infection allows COVID19 to replicate in the human body. Because adaptive immunity, mainly CD8 T-Cells, not able to eliminate COVID19 infected cells, body depends on neutrophils/Cytokine Storm to control the infection, which leads to inflammation and organ (mainly lungs) damage which leads to energy depletion. While differentiating, CD4, CD8 effector T-cells have higher energy needs which fails.

It is not just boosting of immunity or complete reduction of inflammation or restoring energy, these has to be modulated/regulated to suits to each individual need. Modern medicine has all the ability to save millions of lives, but do not have ability to modulate/regulate multiple mechanisms at the same time on continuous basis, as most of the modern science is based on dose dependent up-regulation or down-regulation of one single parameter like fever, pain, antiviral/vaccine attacking spike protein.

For some diseases like HIV, HCV, ALZ, CVD, COVID19, RA, CAN continuous modulation/regulation of immunity-inflammation-energy could be the solution for better health. When integrated medicine chooses both modern medicine and natural products which has capability of this modulation or regulation, that may help humans, mainly aged adults to have better health and longevity.

### DETAILED DESCRIPTION OF INVENTION

Picrorhiza Kurroa, traditionally known as Kutki in Ayurveda for more than 3500 yrs. Picrorhiza Kurroa and its related species are grown in Himalayan regions at higher altitudes. Picrorhiza Kurroa mainly available in India and Bhutan, Neopicrorhiza scrophulariiflora mainly available in China and Picrorhiza scrophulariiflora Pennell mainly available in Nepal and Bhutan. These three species together are referred as “PK Plants” for this invention.

These are known with common names of Picrorhiza, Kutki, Katuka, hu-huang-lian etc

General or commercial usage of these PK Plants are mainly raw root/rhizome in powder form, tea made of that powder or cut pieces(mainly in China), aqueous extract, alcohol extract or Hydro-Alcohol extracts or polyherbal compositions of those(all these together referred as “Traditional Extracts” for this invention). All these contains naturally occurring glycosides like Picroside-I/II/III/IV and Kutkoside, terpenoids and these tastes very bitter after extraction. Though individual tolerance may vary, consumption at higher doses may cause nausea, vomiting and diarrhoea due to its inherited high bitterness.

First named inventor is working for more than a decade in developing process to eliminate drawbacks of Traditional Extracts like bitterness, safety at even very high doses and good for daily & long term consumption. From then he is making continuous improvements and modifications in the process and quality to give better product for human consumption and alleviating human suffering.

Prior art and most of research on PK Plants are mainly based on Traditional Extracts. Some researcher used other solvents but those are mainly for fractioning purpose for in-vitro testing and others used unfractioned Traditional Extracts in in-vivo testing.

Traditional Extract of PK Plants are known for treatment of liver diseases, viral fevers, asthma, Hepatitis etc. Most of other research which took place on immunomodulation, antivirals, vaccine adjuvants, kidney diseases, diabetes, hyperlipidemia, cancer etc could not prove enough to get into main stream of human usage. Main formulation which commercially available is Picroliv developed by CSIR, Govt of India and rest all compositions with Traditional Extract mainly from India and China, with combination of many other herbs and extracts. Traditional Knowledge Database Library (TKDL), India has many references for using of Kutki in traditional medicines like Ayurveda, siddha, unani, but most of those are with combination of something like 20-30 herbs. More over modern solvents like Petroleum Ether, Hexane, n-Hexane, SCCO2 (Super Critical Carbon dioxide) were not available during those ancient times.

Unless we modulate/regulate immunity-inflammation-energy together it may not be possible to give effective and safe treatment to COVID19, CVD, ALZ, Sepsis, Cancers.

Co-inventors(2^{nd} and 3^{rd}) both are doctors by profession and they had to work in hospitals without availability of any proved antiviral or vaccine for them in the initial stages of COVID19, that too with PPE Kits, Face mask, shields for many hours on COVID19 patients/in COVID wards. With the First named inventor prior work and knowledge, they started using HE to improve their immunity and protect themselves. Inspite of getting RT-PCR, Antigen tests positive with mild disease, they could not see COVID19 IgG antibodies. Later research by others shown that T-Cells can protect from COVID19 infection, even if there are not enough antibody response due to virus resolution in short period i.e. before adaptive immunity get activated. As front line COVID19 warriors they worked for almost a year without worrying about COVID19 disease before they got COVID19 Vaccine.

It was surprising finding by the inventors that humans can get protections from Novel Diseases, like COVID19 without even having effective vaccines or IgG antibodies as long as their T-Cells has capability to counter the pathogens. Others Research also shown that's hospitalised patients of SARS-Cov-1 (˜2003) still have T-Cell response to corona viruses and has protection to some extent to SARS-CoV-2 in 2020 i.e. after 17 yrs, whereas antibodies from recent infections are disappearing and needed booster doses for better protection.

First named inventor's tests and research proved HE has capability of modulated/regulate T-Cells in short span of time i.e. even in a week even in immune compromised patients and aged adults.

It is already known that vaccines for aged adults (say 65+ yrs) do not work well due to immune-aging. They need naïve T-Cells and B Cells so that they can generate effective IgG response, which are lacking due to immune senescence.

As of June, 2021 there is no effective anti-viral treatment for COVID19. Antibody cocktails also failing due to mutations. Governments and public mainly dependent on vaccination, steroids, IL6 blockers etc. Recent variants like Delta Plus possibly developed immune escape mechanism and spreading much faster than earlier COVID19 variants/mutants. Major vulnerable population are aged adults and immune compromised, transplant patients or cancer patients. Situation warrants immediate alternative to take care of immunity-inflammation-energy, so that COVID19 warriors like doctors, nurses can save more people under their care.

Following are some of the features inventor has observed/anticipated in the HE:
HE has ability to improve quantity/functionality of Naïve/effector/memory T-cells of mainly CD4 and CD8, even in aged adults or immune compromised
HE has ability to regulate Neutrophils
HE has ability to reduce cellular ROS, possibly by modulating glutathione
HE has ability to trigger CD8 immunity to kill/eliminate infected or mutated cells
HE has ability to modulate TP53 Gene and p53 protein which protects cells
HE has ability to modulate inflammasomes like NLRP3
HE has ability to improve ATP function whereby increasing energy levels, which also needed for better immunity like CD4, CD8 differentiation
HE has ability to modulate CD36 in cancers, liver, cardio, neurodegeneration, metabolic and other diseases
HE has ability to modulate/regulate, but not limited to, miR155, miR223, miR29, miR125 etc.
HE has ability to modulate complement system protein synthesis and response
HE may contain lipid soluble Phospholipids, membrane lipids, exosomes of PK plants.
HE has shown ability to reversing T-Cell exhaustion and causing sustained immune response. This could be due to HE ability to reversing immune aging by increasing length of telomeres.
HE has ability to modulate autophagy, mitochondria and telomerase, and its function, especially in aging.
HE has ability to modulate/regulate Naïve T-Cells in aged adults possibly by reversing Immune senescence i.e. aging of the human immune system to some extent. That enables vaccines for aged adults to work better.
HE can be used as immune-adjuvant in treatment of COVID19 infections, cancers and ALZ etc.
HE is safe at higher doses and good for long term use.

Some of the earlier researches have been done mainly in in-vitro models using Traditional Extracts or fractions isolated for research purpose at temperatures much higher than present invention.

For example Apocynin has many acivities like NADPH Inhibitor, anti-inflammatory, oxidative stress inducer, glutathione modulator is well documented, but it has to be ingested at very high doses to be therapeutically effective. As a chemical molecule, Apocynin alone cannot regulate immunity-inflammation-energy. Whereas HE proved effective in human host at 300-400 mg per day.

HE as an extract has ability to regulate immunity-inflammation-energy which will be beneficial for prevention, management and treatment of COVID19. Especially in aged adults who are getting affected more due to their low energy level, low immunity and chronic inflammation, besides comorbidities.

In COVID19 infection it is noticed that most of the cases (80-90%)are asymptomatic and mild cases which get resolved in less than 7 days , but normally adaptive immunity do not trigger in less than 7 days to create T-Cell response or Antibody response for novel infections. COVID19 vaccines becoming more effective in non-hospitalised cases because most of infection symptoms are less than 7 days, but transmissible upto 14 days. Antibodies can only tag an infected cell, but need effective CD8 response to kill infected cells and stop replication. Need good CD4 response to kill extra cellular pathogens and also to trigger B-Cell response to generate antibodies.

To achieve faster recovery in COVID19 or any future novel diseases we need to balance between immunity-inflammation-energy. For example certain level of inflammation is required for immune activation and repair, but if it exceeds required level it may cause neutrophilia, T-Cell exhaustion and cytokine storm. So balancing is needed for optimal immune function and recovery.

Inventor realised that a speciality botanical extract from Picrorhiza (selected from one or more plants from Picrorhiza kurroa, Neopicrorhiza scrophulariiflora, and Picrorhiza scrophulariiflora Pennell. Collectively called as “PK plants”) can correct immune-dysregulation but cannot reduce the burden of inflammation completely caused by ROS, disease or infection. If acute or chronic inflammation not reduced it causes again immune dysregulation by further causing inflammation and interfering with function of T-Cells and B-Cells generated by Thymus and Bone Marrow. Not able to generate enough naive T-Cells and B-Cells leads to unable to manage disease or infection, as immune system cannot identify and repair cells or guard from new infections, diseases or mutations caused by diseases like cancers. Naïve T-cells are even required to control or elimination of plaques and tangles in ALZ; plaques and blocks in CVD; control and eliminate mutated cells in coordination with p53 in cancers.

To address this issue along with the immune regulation we need to further reduce burden of ROS/Cytokines/Chemokines in the body and brain to effectively prevent, treat or manage diseases. Inventor proposes to add/co-administer therapeutically active doses antioxidants/vitamins like Vitamin C(>2000 mg per day), Glutathione, Q10; and other/reduced forms of those for enhanced efficacy and speedy recover.

Lymphopenia in covid19 affects both CD4 and CD8, though its preferential reduction is of CD8 T-Cells. So correction of Lymphopenia and restoring lymphocytes becomes essential in COVID19 resolution.

Excess Neutrophils recruitment in the lungs and formation of Neutrophil Extracellular Traps (NETs) reported causing lung injury in the COVID19 disease due to high Neutrophil-Lymphocytes-Ratio (N-L-Ratio).

Some researchers observed that Lymphopenia is happing ˜7 day of disease, followed by ARDS and Neutrophilia by 9^{th} day. Possible cause could be failure recruiting adaptive immunity (mainly CD8), which makes more dependency on inflammation and neutrophils for disease resolution. Neutrophilia and formation of NETs may be causing permanent lung damage even in some resolved cases.

Normally severe covid19 patients join hospital in 1-3 days from getting symptoms, which leaves window of 4-5 days to boost lymphocytes and reduction of neutrophils to required level so that patient don't get into cytokine storm. That is where present invention possibly help to regulate lymphocytes and prevent cytokine storm or reduce hospitalisation time.

Preliminary data from the ongoing clinical studies on HE in aged adults who are suffering with mild COVID19 indicates that HE is started to modulate/regulate lymphocytes mainly CD4, CD8 in very short period so that recovery of patients become better.

Composition claimed is expected to modulate miR to generate/regulate T-Cells, B-Cells; and p53 Gene for prevention, management and treatment for Cancers, Neurodegenerative dieses, infections, CVD, vascular & neurovascular diseases and pain management.

Due to its ability to regulate immunity-inflammation-energy at the same time, HE and its formulations would be useful in prevention, management and elimination of diseases or disorders like, but not limited,

Rheumatoid arthritis, Sepsis, ARDS, CVD, Irritable bowel syndrome (IBS), chronic/systemic inflammation

Liver diseases like MAFLD, NASH, ALD, Fibrosis, Cirrhosis, HCC, HCV wherein low immunity-high inflammation makes it difficult to treat

Cancers and Neuroinflammatory diseases like ALZ, Parkinsons disease (PD), Amyotrpic Lateral sclerosis(ALS), Multiple sclerosis(MS) which are known for imbalance in inflammation, immunity and energy metabolism.

Aging related Immunosenescence, low/non-responsiveness to vaccines, improving functionality of innate and adaptive immunity and also improving effectiveness of complement system.

Regulation of T-Cells (mainly CD4 and CD8) and B cells. Also Naïve cells of Lymphocytes.

Correction of functionality of glands like Thymus and Thyroid.

Regulation and correction of cancer suppressor gene like p53 and p53 protein.

Pain management. Mainly for adults where they have chronic and low level inflammation and pain

HE extract of PK Plants is expected to have, but not limited to, following features:
Contains Aglycons of glycosides, glucosides, picrosides, terpenoids of PK Plants
Substantially free from sugar moieties of PK Plants like picrosides, kutkosides, terpenoids etc.
Comprises non-polar components more than 80 pct and bipolar components less than 20 pct.
Of fatty acids in HE of PK plants where in more than 60 pct are non-polar fatty acids and less than 40 pct are bipolar fatty acids.
contains non-coding RNAs like miR (micro RNAs), antisense RNAs of PK Plants and/or has ability to modulate microRNAs.
Contains Vanillic acid, Cinnamic acid, Acetovanillone, Catalpol; and its related compounds
further comprises one or more of the other apolar constituents, exosomes, membranes lipids and degraded molecules of PK Plants
HE is in semi-solid state at room temperature and liquid state between 40-60 deg C.

Following are some of the examples/embodiments of improved extraction process. Any modifications, improvements to this invention done by persons who has skill and art will be in the scope and spirit of this invention and the same are anticipated.

### Embodiment-1

PK Plants roots/rhizomes are collected, cleaned, washed with normal water and dried. This entire process is done below 35 deg C.

Roots/rhizomes are pulvarised to fine to coarse powder

Powder is loaded into extractor in the measured quantity

Extraction process is conducted with Super Critical CO2(SCCO2) at temperatures below 35 deg C. and higher pressures needed.

HE is collected in collection vessel and sent for testing and formulation.

### Embodiment-2

PK Plants roots/rhizomes are collected, cleaned, washed with normal water and dried. This entire process is done below 15 deg C.

Roots/rhizomes are pulvarised to fine to coarse powder and stored below 15 deg C.

Powder is loaded into extractor in the measured quantity

Extraction process is conducted with Super Critical CO2(SCCO2) and with co-solvent at temperatures below 35 deg C. Co-solvent is selected from hexane, n-hexane or any other suitable apolar solvent.

HE is collected in collection vessel and sent for further process to removal of co-solvent. Co-solvent is removed below 25 deg C. with ˜1 atm pressure.

HE is collected and sent for testing and formulation.

### Embodiment-3

PK Plants roots/rhizomes are collected, cleaned, washed with clean water and dried to below 15% moisture content. This entire process is done below 25 deg C., more preferably 15 deg C.

Roots/rhizomes are pulverised to fine to coarse powder below 15 deg C. and stored below 15 deg C. in vacuum sealed containers

Powder is loaded into extractor in the measured quantity

Extraction process is conducted using solvent at ˜1 atm pressure, below room temperature. Where in solvent is selected from hexane, n-hexane, Petroleum Ether 30-60 deg C. or any other suitable apolar solvent

HE+solvent is collected in collection vessel and sent for further process to remove solvent. Solvent is removed in regulated air/nitrogen/oxygen/hydrogen atmosphere below room temperature, at ˜1 atm pressure. Where in core temperature of the HE during separation should not exceed 12.5 deg C.

HE thus collected is in semisolid condition, sent for testing and formulation in controlled conditions.

### Embodiment-4

PK Plants roots/rhizomes are collected, cleaned, washed with normal water and dried. This entire process is done below 25 deg C.

Roots/rhizomes are pulvarised to fine to coarse powder at below 25 deg C. and stored below 25 deg C. in vacuum sealed containers

Powder is loaded into extractor in the measured quantity

Extraction process is conducted using solvent at temperatures below 35 deg C. at ˜1 atm pressure, where in solvent is selected from Petroleum Ether 30-60 deg C., more preferably Petroleum Ether 40-60 deg C. or any other suitable apolar solvent with low boiling point.

HE+solvent is collected in collection vessel and sent for further process to remove solvent. Solvent is removed at temp below 25 deg C. with ˜1 atm pressure while making sure that any given point HE contact temp is maintained below 12.5 deg C. with ˜1 atm pressure.

HE thus collected sent for testing and formulation in controlled conditions.

Wherever it is mentioned as roots/rhizomes in this application, it is understood that it meant roots and/or rhizomes of PK Plants.

This improved extraction process and technology may be used for other plant extracts like, but not limited to, Neem, Curcumin, Tulasi, Ashwagandha, ginseng, and the same is anticipated by the inventors and falls under scope of this invention.

### REFERENCES

1. Stefanska J, Pawliczak R. Apocynin: molecular aptitudes. Mediators Inflamm. 2008; Epub 2008 Dec. 2.

2. TUHIN KANTIBISWAS et al, EFFECT OF PICRORHIZA KURROA BENTH. IN ACUTE INFLAMMATION, Ancient Science of life Vol No XVI I July 1996, Page 11-14

3. Johansson, C., Kirsebom, F. C. M. Neutrophils in respiratory viral infections. Mucosal Immunol 14, 815-827 (2021)

4. Marjolein Kikkert, Innate Immune Evasion by Human Respiratory RNA Viruses, Innate Immun 2020;12:4-20

5. www.who.int/docs/default-source/coronaviruse/risk-comms-updates/update-34-immunity-2nd.pdf

6. Rosales C. Neutrophil: A Cell with Many Roles in Inflammation or Several Cell Types?. Front Physiol. 2018; 9:113. Published 2018 Feb. 20.

7. Le Bert, N., Tan, A. T., Kunasegaran, K. et al. SARS-CoV-2-specific T cell immunity in cases of COVID-19 and SARS, and uninfected controls. Nature 584, 457-462 (2020).

8. Diao B, et al Reduction and Functional Exhaustion of T Cells in Patients With Coronavirus Disease 2019 (COVID-19). Front. Immunol. 2020:11:827.

9. Tay, R. E., Richardson, E. K. & Toh, H. C. Revisiting the role of CD4⁺T cells in cancer immunotherapy-new insights into old paradigms. Cancer Gene Ther 28, 5-17 (2021).

1-20. (canceled)

21. A method of preparing a hydrophobic extract, comprising:
obtaining a powder from roots/rhizomes of PK plants in which the PK plants have been collected, cleaned, washed, and dried at a temperature below 25° C. to obtain dried PK plants and the dried PK plants are pulverized into the powder at a temperature below 15° C.; and
preparing the hydrophobic extract by performing extraction on the powder with an apolar solvent and/or super critical carbon dioxide at a temperature below 35° C.,
wherein the PK plants comprise Picrorhiza kurroa, Neopicrorhiza scrophulariiflora, Picrorhiza scrophulariiflora, or any combination thereof.

22. The method of claim 21, wherein:
the hydrophobic extract comprises more than 80% by weight of nonpolar components and less than 20% by weight of polar/bipolar components; and
the hydrophobic extract is in a liquid state between 40° C. and 60° C.

23. The method of claim 21, wherein the hydrophobic extract comprises aglycons of glycosides, glucosides, picrosides, and terpenoids of the PK plants.

24. The method of claim 21, wherein the hydrophobic extract is substantially free from sugar moieties of the PK plants.

25. The method of claim 21, wherein:
the hydrophobic extract comprises fatty acids of the PK plants;
more than 60% by weight of the fatty acids are nonpolar fatty acids; and
less than 40% by weight of the fatty acids are polar/bipolar fatty acids.

26. The method of claim 21, wherein the hydrophobic extract comprises non-coding RNAs.

27. The method of claim 21, wherein the hydrophobic extract has an ability to modulate microRNAs.

28. The method of claim 21, further comprising formulating a composition comprising the hydrophobic extract and one or more of vanillic acid, cinnamic acid, acetovanillone, or catalpol.

29. The method of claim 21, wherein the hydrophobic extract comprises cell membranes and exosomes of the PK plants.

30. The method of claim 21, further comprising formulating a composition comprising the hydrophobic extract wherein the composition is formulated as a powder, syrup, drink, tablet, caplet, softgel, capsule, nanogel, nano-particles, injection, parenteral, transdermal patch, absorbent gel, nasal spray, vaginal gel, or gel strip.

31. The method of claim 21, further comprising formulating a composition comprising the hydrophobic extract wherein the composition is formulated as an eye drop.

32. The method of claim 21, further comprising formulating a composition comprising the hydrophobic extract wherein the composition is adsorbed on an excipient.

33. The method of claim 21, further comprising formulating a composition comprising the hydrophobic extract wherein the composition is in a form suitable for administration through an oral, intravenous, intramuscular, intravesical, sub-cutaneous, peritoneal, rectal, nasal, trans-dermal, dermal, ophthalmic, sublingual, vaginal, or buccal route.

34. The method of claim 21, further comprising formulating a composition comprising the hydrophobic extract wherein the composition comprises 50 mg to 5000 mg of the hydrophobic extract.

35. The method of claim 21, wherein:
the extraction is performed with the apolar solvent;
the apolar solvent is hexane, n-hexane, or petroleum ether (Grade 30-60° C.);
the apolar solvent is removed at a temperature below 25° C. and at a pressure of about 1 ATM pressure; and
a temperature of the hydrophobic extract is maintained not above 12.5° C. during removal of the apolar solvent.

36. The method of claim 21, wherein the extraction is performed with the super critical carbon dioxide.

37. The method of claim 21, wherein the extraction is performed with the apolar solvent and the super critical carbon dioxide.

38. A hydrophobic extract prepared by the method of claim 21.

39. A method for prevention, management, and/or treatment of Immune Dysregulation, Inflammation Dysregulation, and/or Energy Dysregulation by modulation/regulation of ncRNAs, Lymphocytes, Neutrophils, Neutrophils-Lymphocytes-Ratio (NLR), Inflammasomes, p53, IL-6, TNF-Alpha and/or reactive oxygen species (ROS) comprising administering an effective amount of a composition comprising the hydrophobic extract of claim 21 to a subject in need thereof.

40. The method of claim 39, wherein the composition is co-administered with 2000 mg or more of ascorbic acid.
